(19) United States
(12) Patent Application Publication
(10) Pub. No.: US 2007/0012316 A1
(43) Pub. Date: Jan. 18, 2007

(54) Disposable compact rescue inhaler

(76) Inventor: Joann Truza, Troy, MI (US)

Correspondence Address: BLISS MCGLYNN, P.C., 2075 WEST BIG BEAVER ROAD, SUITE 600, TROY, MI, 48084, US

(21) Appl. No.: 11/487,116

(22) Filed: Jul. 14, 2006

Related U.S. Application Data
(60) Provisional application No. 60/699,224, filed on Jul. 14, 2005.

(51) Int. Cl. A61M 11/00

(52) U.S. Cl. 128/200.23

(57) ABSTRACT

A disposable and compact rescue inhaler including a package containing pressurized fluid medicament for treating respiratory constriction and a housing having a plurality of walls that cooperate to define an internal cavity to store the package. At least one of the walls includes an outlet to expel the medicament in an aerosolized form to a user. The housing also includes a passage between the outlet and the internal cavity to deliver the medicament to the outlet. At least one of the walls includes an inlet and the housing includes a channel disposed between the inlet and the passage to direct ambient air into the passage to facilitate aerosolizing the medicament. The inhaler further includes an attachment member and an actuator disposed to operatively engage the package and dispense medicament into the passage. The inhaler further includes a removable cover plate to prevent debris from infiltrating the inlet and the outlet.

### CROSS-REFERENCE TO RELATED APPLICATION(S)

This application claims the benefit of U.S. Provisional Patent Application Ser. No. 60/699,224 entitled, “Disposable Compact Rescue Inhaler,” filed on Jul. 14, 2005.

### BACKGROUND OF THE INVENTION

1. Field of the Invention

The present invention relates, generally, to a portable inhaler for dispensing an intrapulmonary medicament. More specifically, the present invention relates to a disposable and compact rescue inhaler for administering a pressurized liquid medicament in aerosolized form.

2. Description of the Related Art

Inhalers are commonly employed to administer intrapulmonary medicaments to persons having asthma or other respiratory ailments. Some inhalers are intended for stationary use while others are portable. Generally, there are two types of portable inhalers for administering an intrapulmonary medicament. Each of these types of inhalers provide a metered dose of medicament and, thus, are commonly referred to as “Metered Dose Inhalers” or “MDIs.” One type of inhaler involves administering a powdered form of medicament that prevents the occurrence of an asthma attack. Since the user's airway is not constricted, the powdered form medicament is delivered to the respiratory system as the user inhales, thereby pulling the medicament into the user's lungs.

The other type of portable MDI is designed to provide relief during an asthma attack. Due to the constriction of the respiratory system during an asthma attack, it is difficult to inhale the medicine, as performed with respect to the first method of treatment. As a result, the second treatment method involves administering a metered dosage (approximately 90 mcg) of medicament, typically in liquid form, with the assistance of a propellant, such as a fluorocarbon or other pressurized gas, to project aerosolized medicament into the respiratory system and to relax the constricted area. Since this second method of treatment is designed for use during constriction of the respiratory system, such as during an asthma attack, it is commonly referred to as a “rescue inhaler.”

It is strongly encouraged that persons with respiratory ailments such as asthma keep a rescue inhaler readily available at all times, since an asthma attack may occur at any moment. However, rescue inhalers of the type typically known in the art include an L-shaped housing and a pressurized canister containing approximately 200 metered doses of medicament. As a result, typical rescue inhalers are often too bulky (i.e. 7.65 inches of cubic area) and cumbersome to be readily available, especially when participating in athletic activities or during occasions where one's attire limits the amount of available pocket/carrying space. In these situations, an asthmatic person may choose not to carry a rescue inhaler, which can have life-threatening consequences.

As a result, there is a need in the art for a compact rescue inhaler capable of delivering a metered dose of pressurized medicament that is readily available at all times. Accordingly, there is a need in the art for a compact and disposable rescue inhaler that delivers a metered dose of pressurized medicament for relieving respiratory constriction yet having a substantially smaller profile than traditional rescue inhalers. Furthermore, there is a need in the art for a compact and disposable rescue inhaler that may be operatively disposed on a person during essentially all occasions.

### SUMMARY OF THE INVENTION

The present invention overcomes many limitations and disadvantages in the related art in metered dose rescue inhalers to provide a disposable compact rescue inhaler to dispense pressurized fluid medicament in an aerosolized form. To this end, the compact and disposable rescue inhaler of the present invention includes a package containing a pressurized fluid medicament for treating respiratory constriction and a housing having a plurality of walls. The walls of the housing cooperate to define an internal cavity therein that is adapted to store the package. At least one of the walls includes an outlet to expel the pressurized fluid medicament in an aerosolized form to a user. One of the walls also includes a passage disposed between the outlet and the internal cavity to facilitate delivery of the aerosolized medicament toward the outlet. At least one of the walls further includes at least one inlet defined therein. The housing further includes at least one channel disposed between the inlet and the passage. The inlet and the channel are adapted to direct ambient air into the passage to facilitate aerosolizing the pressurized fluid medicament. The compact and disposable rescue inhaler further includes an attachment member operatively attached to the housing to facilitate removable attachment of the housing to a person. The compact and disposable rescue inhaler further includes an actuator disposed within one of the walls that is adapted to operatively engage the package to dispense pressurized fluid medicament into the passage. The compact and disposable rescue inhaler further includes a removable cover plate adapted to fit over a portion of the housing to prevent debris from infiltrating the inlet and the outlet.

In another embodiment the compact and disposable rescue inhaler of the present invention includes a canister-style package containing a pressurized fluid medicament for treating respiratory constriction and a housing having a substantially cylindrical body and a mouthpiece depending therefrom. The cylindrical body has an internal cavity defined therein that is adapted to store the canister-style package. The mouthpiece further includes an outlet to expel the pressurized fluid medicament in an aerosolized form to a user and a passage disposed between the outlet and the internal cavity to facilitate delivery of the aerosolized medicament toward the outlet. The mouthpiece also includes at least one inlet defined therein and at least one channel disposed between the inlet and the passage to direct ambient air into the passage to facilitate aerosolizing the pressurized fluid medicament. According to this embodiment, the compact and disposable rescue inhaler further includes an attachment member operatively attached to the body to facilitate removable attachment of the housing to a person and an actuator disposed within the body. The actuator is adapted to operatively engage the canister-style package to dispense pressurized fluid medicament into the passage. The compact disposable rescue inhaler further includes a removable cover plate adapted to fit over a portion of the mouthpiece to prevent debris from infiltrating the inlet and the outlet.

Thus, one advantage of the present invention is that it provides a compact and disposable rescue inhaler having a housing that is substantially smaller than traditional rescue inhalers to provide pressurized fluid medicament for treating respiratory ailments and that is readily available to persons afflicted by such ailments during all types of activities.

Another advantage of the present invention is that it provides a compact and disposable rescue inhaler that provides a metered dose of medicament that is greater than 90 mcg to enable the pressurized fluid medicament to penetrate the affected area within a person's respiratory system.

Still another advantage of the present invention is that it provides a compact and disposable rescue inhaler that is adapted to provide readily accessible medicament to enable persons afflicted with respiratory ailments additional time to access other respiratory treatments, if necessary, such as during an acute respiratory ailment attack.

Other objects, features and advantages of the present invention will be readily appreciated as the same becomes better understood after reading the subsequent description in connection with the accompanying drawings.

### BRIEF DESCRIPTION OF THE DRAWINGS

FIG. 1 is a perspective view of one embodiment of the present invention.

FIG. 2 is a cut-away perspective view of the embodiment of the present invention illustrated in FIG. 1.

FIG. 3 is a perspective view of another embodiment of the present invention.

FIG. 4 is a cross-sectional top view of the embodiment of the present invention illustrated in FIG. 3.

### DETAILED DESCRIPTION OF THE PREFERRED EMBODIMENT(S)

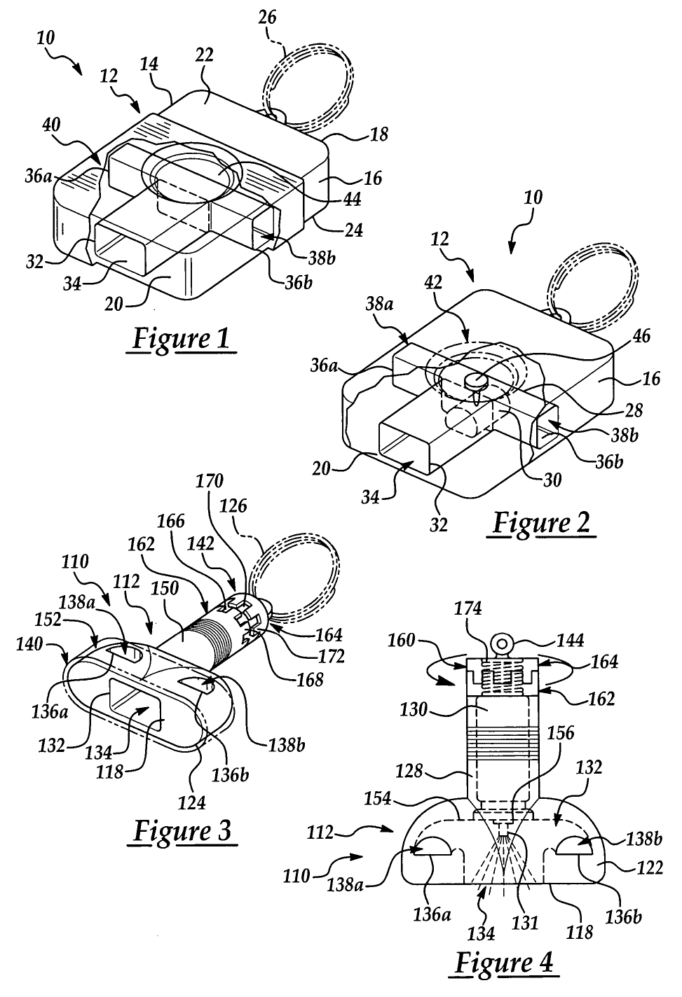

Referring now to FIGS. 1 and 2, a rescue inhaler of the present invention is generally indicated at 10. The rescue inhaler 10 provides a compact and disposable inhaler that is effective for administering a metered dose of fluid medicament in aerosolized form. The inhaler 10 includes a housing, generally indicated at 12. The housing 12 is adapted to protect and store remaining components of the inhaler 10. The housing 12 is defined by a plurality of walls. More specifically, the housing includes first and second sidewalls 14 and 16, respectively, rear and front walls 18 and 20, respectively, and upper and lower walls 22 and 24, respectively. It should be appreciated by those having ordinary skill in the art that the housing 12 is substantially compact when compared to traditional rescue inhalers. By way of example, the housing 12 of the present invention is approximately the size of a vehicle key fob transmitter or approximately three inches long by two inches wide by three-quarters of an inch thick or smaller. The housing 12 is constructed from a rigid-forming polymer material and may be manufactured via injection molding, cast molding or other methods commonly employed in the art. The inhaler 10 further includes an attachment member 26 operatively attached to the housing 12 to facilitate removable attachment of the inhaler 10 to a person. As shown in FIG. 1, the attachment member 26 is a key ring. However, those having ordinary skill in the art will appreciate that the attachment member 26 may be any other structure adapted to facilitate such attachment. By way of example, the attachment member 26 may be a clip, belt, strap or necklace to achieve the intended objective.

Referring to FIGS. 1 and 2, the walls of the housing 12 cooperate to define an internal cavity 28. The inhaler 10 further includes a package 30 that contains pressurized fluid medicament for treating respiratory constriction. The internal cavity 28 is adapted to store the package 30. As shown in FIG. 2, the package 30 is operatively disposed within the internal cavity 28. It should be appreciated that the package 30 is fixed (i.e. non-removable) within the internal cavity 28. The package 30 disposed within the internal cavity 28 is a blister-style package. The blister-style package accommodates a predetermined volume of fluid medicament for treating various respiratory ailments, such as Albuterol, as well as a pressurized propellant for delivering the medicament, such as a chlorofluorocarbon (“CFC”). The volume and composition of fluid medicament is predicated upon the amount desired to treat a particular respiratory ailment. However, it should be appreciated that where the medicament is Albuterol, the volume within the blister-style package is greater than a standard metered dose received through MDI rescue inhalers commonly known in the art, i.e., 90 mcg. Those having ordinary skill in the art will readily appreciate that the inhaler 10 illustrated in FIGS. 1 and 2 is not intended to replace typical MDI rescue inhalers, but rather to be employed in connection therewith. Specifically, the inhaler 10 is adapted to provide immediate relief from an attack and enable a person to access another other treatment device for respiratory ailments, if necessary. Accordingly, the package 30 disposed within the inhaler 10 illustrated in FIGS. 1 and 2 of the present invention is adapted to provide a single dosage of pressurized fluid medicament between 130-270 micrograms (“mcg”), preferably between 130-180 mcg. More specifically, the inhaler 10 provides a single-dose of pressurized fluid medicament for a predetermined length of time that is sufficient to enable the user at least two consecutive inhales.

The inhaler 10 further includes an outlet 32 defined within one of the walls. As shown in FIGS. 1 and 2, the outlet 32 is defined within the front wall 20. The outlet 32 is adapted to expel the pressurized fluid medicament in an aerosolized form to a user. The inhaler 10 further includes a passage, generally indicated at 34. The passage 34 is disposed between the internal cavity 28 and the outlet 32 and adapted to facilitate delivery of a dosage of the aerosolized medicament toward the outlet 32. Those having ordinary skill in the art will appreciate that while the inhaler 10 illustrated in FIGS. 1 and 2 includes only one outlet 32 and passage 34, the inhaler 10 of the present invention may include any number of outlets connected to a single passage or multiple outlets and passages to accomplish delivery of the medicament to the user.

The inhaler 10 further includes at least one inlet 36 defined within at least one of the walls of the housing 12 and at least one channel, generally indicated at 38, disposed between the inlet 36 and the passage 34. As illustrated in FIGS. 1 and 2, the inhaler 10 includes two inlets 36a and 36b defined within the first and second sidewalls 14 and 16, respectively, as well as two cooperating channels 38a and 38b. The inlets 36a and 36b cooperate with their respective channels 38a and 38b to direct ambient air into the passage 34 to facilitate aerosolizing the pressurized fluid medicament as it is directed toward the user. However, those having ordinary skill in the art will appreciate that the inhaler 10 may include any number of inlets and channels suitable for effectively aerosolizing a medicament. By way of example, the inhaler 10 may include only one inlet and one channel. Further, those having ordinary skill in the art will appreciate that while the inlets 36a, 36b and channels 38a, 38b are shown for use in connection with two sidewalls 14, 16, these inlets 36a, 36b and passages 38a, 38b may be defined within a single wall, such as a sidewall 16, back wall 18, upper wall 22 or lower wall 24 to direct ambient air into the passage 34 and further aerosolize the medicament.

The inhaler 10 further includes a cover plate, generally indicated at 40. The cover plate 40 is adapted to fit over a portion of the housing 12 to prevent debris from infiltrating the passage 34 and the channels 38a and 38b when the inhaler 10 is not in use. The cover plate 40 is a molded article constructed from a resilient material such as rubber or plastic and is adapted to removably fit over the outlet 32 and the inlets 36a and 36b. The cover plate 40 may further be adapted to fit over the actuator, as will be described in greater detail below. Those having ordinary skill in the art will appreciate that while the cover plate 40 is illustrated as a molded article, the cover plate 40 may include any practical form to prevent infiltration of debris. By way of example, the cover plate may include an adhesive-backed transparent seal to be peeled off prior to dispensing the medicament. More specifically, the cover plate 40 may include a single-use, disposable transparent seal that is adapted to be peeled off prior to actuating delivery of the pressurized fluid medicament into the passage 34.

With continuing reference to FIGS. 1 and 2, the inhaler 10 further includes an actuator, generally indicated at 42, disposed within the upper wall 22 of the housing 12. The actuator 42 is adapted to operatively engage the medicament package 30 and dispense pressurized fluid medicament into the passage 34. The actuator 42 includes a button 44 to be manually activated for delivery of the medicament. To prevent unintentional delivery of the medicament the button 44 is recessed within the upper wall 22 of the housing 12. Those having ordinary skill in the art will appreciate that the actuator 42 may include structure other than a button 44 that may be manually activated to deliver the medicament. By way of example, the actuator 42 may include a switch or a pull cord.

Referring specifically to FIG. 2, the actuator further includes an engaging device 46 to directly engage the package 30 and effect delivery of the medicament. As shown, the engaging device 46 is a piercing member that is adapted to puncture the blister-style package to provide a port through which the package would dispense pressurized fluid medicament into the passage 34. Release of the pressurized medicament into the passage 34 will mix with the ambient air drawn into the passage 34 from the channels 38a and 38b to further aerosolize the medicament and facilitate delivery through the outlet 32. Those having ordinary skill in the art will appreciate that while a piercing member is employed as the engaging member 46 to engage the package 30, the inhaler 10 of the present invention may include other structure to directly engage the package 30. By way of example, the engaging member 46 may include a wedge-shaped article that crushes a portion of the package 30 when a user depresses the button 44. Furthermore, those having ordinary skill in the art will appreciate that the actuator 42 may include additional structure, such as a notice mechanism to readily identify that the medicament has been dispensed and the inhaler 10 should be disposed of and replaced. By way of example, where a button 44 is employed as a component of the actuator 42, such a notice mechanism may be included within the button 44 wherein the button changes color or is crushable (i.e. unable to be repeatedly depressed). Further by way of example, where the actuator includes a pull cord-type component, such a notice mechanism may be included where the pull cord-type actuator separates from the inhaler 10 upon initiating delivery of the medicament (M).

Another embodiment of the present invention is generally indicated at 110 in FIGS. 3 and 4 where like numerals, increased by 100 with respect to the embodiment of the invention illustrated in FIGS. 1 and 2, are used to designate like structure. The inhaler 110 of the present invention as shown in FIGS. 3 and 4 includes some similar structure to the inhaler 10 shown in FIGS. 1 and 2 albeit in a different configuration. Accordingly, the description of the structure of the inhaler 110 will not be repeated here except where expressly mentioned below.

The inhaler 110 includes a housing, generally indicated at 112. However, unlike the housing 12 illustrated in FIGS. 1 and 2, the housing 112 of this embodiment includes a substantially cylindrical body 150 and a mouthpiece, generally indicated at 152, depending from the body 150. It should be readily appreciated by those having ordinary skill in the art that the housing 112 of the present invention is substantially compact when compared to traditional rescue inhalers. By way of example, the housing 112 of the present invention is approximately the cubic area of a vehicle key fob transmitter or a cubic area of 4.5 inches or smaller. The housing 112 is constructed from a rigid-forming polymer material and may be manufactured via injection molding, cast molding or other methods commonly employed in the art. The inhaler 110 further includes an attachment member 126 operatively attached to the body 150 to facilitate removable attachment of the inhaler 110 to a person. As shown in FIG. 3, the attachment member 126 is a key ring. However, those having ordinary skill in the art will appreciate that the attachment member 126 may any other structure adapted to facilitate such attachment such as that described with respect to the attachment member 26 illustrated in FIGS. 1 and 2.

Referring to FIGS. 3 and 4, the body 150 includes an internal cavity 128 defined therein. The inhaler 110 further includes a package 130 that contains pressurized fluid medicament for treating respiratory constriction. The internal cavity 128 is adapted to store the package 130. As shown in FIG. 4, the package 130 is operatively disposed within the internal cavity 128. It should be appreciated that the package 130 is fixed (i.e non-removable) within the internal cavity 128. The package 130 disposed within the internal cavity 128 is a canister-style package. The canister-style package accommodates a predetermined volume of fluid medicament for treating various respiratory ailments, such as Albuterol, as well as a pressurized propellant for delivering the medicament, such as a chlorofluorocarbon (“CFC”). Specifically, the canister-style package includes a nozzle 131 that expels medicament when depressed.

The volume and composition of fluid medicament is predicated upon the amount desired to treat a particular respiratory ailment. However, it should be appreciated that where the medicament is Albuterol, the volume within the canister-style package is adapted to provide metered doses that are greater than a standard metered dose traditionally received through MDI rescue inhalers commonly known in the art, i.e., 90 mcg. Accordingly, the package 130 disposed within the inhaler 110 illustrated in FIGS. 3 and 4 is adapted to contain between 380 and 460 mcg of pressurized fluid medicament to provide at least four metered doses of medicament at over 90 mcg per dose.

The mouthpiece 152 is defined by a front wall 118, upper and lower walls 122 and 124, respectively, and a back plate 154. The back plate 154 is adapted to partition the internal cavity 128 from the passage, as described in greater detail below. The back plate 154 includes an aperture 156 to receive the nozzle 131 of the canister-style package 130. The mouthpiece 152 further includes an outlet 132 defined within the front wall 118 that is adapted to expel the pressurized fluid medicament in an aerosolized form to a user. The mouthpiece 152 further includes a passage, generally indicated at 134, that is disposed between the internal cavity 128 and the outlet 132 and further defined by the back plate 154. The passage 134 is adapted to facilitate delivery of a dosage of the aerosolized medicament toward the outlet 132. Those having ordinary skill in the art will appreciate that while the inhaler 110 illustrated in FIGS. 3 and 4 includes only one outlet 132 and passage 134, the inhaler 110 may include alternative structure as described with respect to the inhaler 10 illustrated in FIGS. 1 and 2.

The mouthpiece 152 further includes at least one inlet 136 defined within the top wall 122 and at least one channel, generally indicated at 138, disposed between the inlet 136 and the passage 134. As illustrated in FIGS. 3 and 4, the inhaler 110 includes two inlets 136a and 136b defined within the top wall 122 as well as two cooperating channels 138a and 138b. The inlets 136a and 136b cooperate with their respective channels 138a and 138b to direct ambient air into the passage 134 to facilitate aerosolizing the pressurized fluid medicament as it is directed toward the user. However, those having ordinary skill in the art will appreciate that the inhaler 110 may include any number of inlets and channels suitable for effectively aerosolizing a medicament.

The inhaler 110 further includes a cover plate, generally indicated at 140. The cover plate 140 is adapted to fit over a portion of the mouthpiece 152 to prevent debris from infiltrating the passage 134 and the channels 138a and 138b when the inhaler 110 is not in use. The cover plate 140 is a transparent seal that is adapted to be peeled off prior to actuating delivery of the pressurized fluid medicament into the passage 134. Those having ordinary skill in the art will appreciate that while the cover plate 140 is illustrated in FIG. 3 is a peel-off seal, the inhaler 110 may include a cover plate 40 as described with respect to the inhaler 10 illustrated in FIGS. 1 and 2. More specifically, the cover plate 140 may include a molded article constructed from a resilient material such as rubber or plastic that is adapted to removably fit over the outlet 132 and the inlets 136a and 136b.

The inhaler 10 further includes an actuator, generally indicated at 142, disposed at the terminal end of the housing body 150. The actuator 142 is adapted to initiate dispensing the medicament. Referring specifically to FIG. 4, the actuator 142 includes a button 144 positioned to operatively engage the canister-style package 130. When manually engaged, the button 144 forces the package 130 against the back wall 154, thereby causing the nozzle to expel the medicament into the passage 134. Release of the pressurized medicament into the passage 134 will mix with the ambient air drawn into the passage 134 from channels 138a and 138b to further aerosolize the medicament and facilitate delivery.

As shown in FIGS. 3 and 4, the actuator 142 further includes a locking mechanism, generally indicated at 160 that prevents premature or unintentional dispensing of the medicament. The locking mechanism 160 includes a base, generally indicated at 162, and a platform, generally indicated at 164, that are substantially defined within the body 150. The platform 164 is rotatable relative to the base 162. The base 162 includes a plurality of notches 166 and teeth 168 and the platform includes a plurality grooves 170 and extensions 172 that correspond with the notches 166 and teeth 168 to permit or prevent the actuator from engaging the canister-style package. By way of example, the locking mechanism 160 prevents actuation where the teeth 168 are aligned with the extensions 172. However, where the platform 164 is rotated relative to the base 162 such that the extensions 172 align with the notches 166, the actuator may operatively engage the canister-style package 130. More specifically, the actuator includes a biasing member 174 that is operatively disposed between the button 144 and the package 130 that, when compressed, directs the package 130 against the back plate 154, thereby causing the nozzle 131 to expel pressurized fluid medicament into the passage 134.

Furthermore, those having ordinary skill in the art will appreciate that the actuator 142 may include additional structure, such as a notice mechanism to readily identify that the medicament has been dispensed and the inhaler 110 should be disposed of and replaced. Such a notice mechanism may include having a numerical sequence on the teeth 168 to indicate when the last metered dose of medicament has been expelled. Another such notice mechanism may include break-away teeth within the locking mechanism 160 that prevents achieving a locked position once the platform 164 is rotated beyond a predetermined amount relative to the base 162.

The present invention provides a disposable and compact rescue inhaler that is substantially more compact than traditional rescue inhalers (i.e. 4.5 versus 7.65 inches of cubic area) adapted to provide immediately accessible pressurized fluid medicament to provide treatment to persons afflicted with respiratory ailments as well as additional time to access another respiratory treatment device, when necessary, such as during an acute respiratory ailment attack. The inhaler 10, 110 of the present invention further provides a removable cover plate 40, 140 that prevents infiltration of debris when the inhaler 10, 110 is not in use. Furthermore, the disposable and compact rescue inhaler 10, 110 of the present invention provides an attachment member 26, 126 for attachment to persons afflicted with respiratory ailments during all types of activities.

The invention has been described in an illustrative manner. It is to be understood that the terminology which has been used is intended to be in the nature of words of description rather than of limitation. Many modifications and variations of the invention are possible in light of the above teachings. Therefore, within the scope of the appended claims, the invention may be practiced other than as specifically described.

What is claimed is:

1. A disposable and compact rescue inhaler for administering fluid medicament in aerosolized form, said inhaler comprising:
a package containing pressurized fluid medicament for treating respiratory constriction;
a housing having an internal cavity defined therein that is adapted to store said package, said housing defined by a plurality of walls wherein at least one of said walls includes an outlet to expel the pressurized fluid medicament from said package in aerosolized form, said housing having a passage disposed between said outlet and said internal cavity to facilitate delivery of the aerosolized medicament toward said outlet, at least one of said walls having at least one inlet defined therein and said housing having at least one channel disposed between said inlet and said passage, said inlet and said channel adapted to direct ambient air into said passage to facilitate aerosolizing the pressurized fluid medicament;
an attachment member operatively attached to said housing and adapted to facilitate removable attachment of said housing to a person;
an actuator disposed within said housing and adapted to operatively engage said package to dispense the pressurized fluid medicament into said passage; and
a removable cover plate adapted to fit over a portion of said housing to prevent debris from infiltrating said inlet and said outlet.

2. The disposable and compact rescue inhaler as set forth in claim 1 wherein said removable cover plate conceals said actuator to prevent dispensing the pressurized fluid medicament into said passage when said inhaler is not in use and is adapted to be removed from and reinstalled onto said housing to prevent debris infiltration when said inhaler is not in use.

3. The disposable and compact rescue inhaler as set forth in claim 1 wherein said cover plate is a single-use, disposable seal adapted to be peeled off prior to actuating delivery of the pressurized fluid medicament into said passage.

4. The disposable and compact rescue inhaler as set forth in claim 1 wherein said actuator includes a button recessed within one of said walls and an engaging device that directly engages said package, said button is adapted to be manually actuated to direct said engaging device to effect delivery of the pressurized fluid medicament into said passage.

5. The disposable and compact rescue inhaler as set forth in claim 1 wherein said actuator includes a button and biasing member operatively disposed between said button and said package to manually actuate delivery of the pressurized fluid medicament into said passage.

6. The disposable and compact rescue inhaler as set forth in claim 1 wherein said actuator includes a locking mechanism having a base and platform substantially defined within said housing, said platform adapted to be rotated relative to said base to selectively permit said actuator to engage said package to prevent unintentional dispensing of the pressurized fluid medicament.

7. The disposable and compact rescue inhaler as set forth in claim 1 wherein said package includes a blister-style container having at least one predetermined metered dosage of the pressurized fluid medicament between 130-270 mcg.

8. The disposable and compact rescue inhaler as set forth in claim 1 wherein said package includes a canister-style container having between 380 and 460 mcg of the pressurized fluid medicament to provide at least four metered doses of the medicament at over 90 mcg per dose.

9. The disposable and compact rescue inhaler as set forth in claim 1 wherein said housing includes an upper wall, a lower wall, a front wall, a back wall, and first and second sidewalls, said actuator operatively disposed within said upper wall, a first inlet of said at least one inlet disposed within said first sidewall and a second inlet of said at least one inlet disposed within said second sidewall.

10. The disposable and compact rescue inhaler as set forth in claim 1 wherein said housing includes a body and a mouthpiece depending from said body, said body including said internal cavity and said mouthpiece including said inlet, said channel, said passage and said outlet to deliver the pressurized fluid medicament in aerosolized form.

11. The disposable and compact rescue inhaler as set forth in claim 1 wherein said attachment member is a key ring.

12. A disposable and compact rescue inhaler for administering fluid medicament in aerosolized form, said inhaler comprising:
a canister-style package containing pressurized fluid medicament for treating respiratory constriction having a nozzle to dispense the pressurized fluid medicament;
a housing having a substantially cylindrical body and a mouthpiece depending therefrom, said cylindrical body having an internal cavity defined therein that is adapted to store said canister-style package, said mouthpiece including an outlet to expel the pressurized fluid medicament in aerosolized form to a user and a passage disposed between said outlet and said internal cavity to facilitate delivery of the aerosolized medicament toward said outlet;
an attachment member operatively attached to said body and adapted to facilitate removable attachment of said housing to a person;
an actuator disposed within said body and adapted to operatively engage said canister-style package to dispense the pressurized fluid medicament into said passage; and
a removable cover plate adapted to fit over a portion of said mouthpiece to prevent debris from infiltrating said inlet and said outlet,
wherein said mouthpiece further includes at least one inlet defined therein and at least one channel disposed between said inlet and said passage, said inlet and said channel adapted to direct ambient air into said passage to facilitate aerosolizing the pressurized fluid medicament.

13. The disposable and compact rescue inhaler as set forth in claim 12 wherein said mouthpiece further includes a back plate that substantially partitions said passage from said internal cavity, said back plate adapted to operatively engage a portion of said canister-style container and having an aperture adapted to receive said nozzle.

14. The disposable and compact rescue inhaler as set forth in claim 12 wherein said canister-style package includes a volume of the pressurized fluid medicament between 380 and 460 mcg to provide at least four metered doses of the medicament at over 90 mcg per dose.

15. The disposable and compact rescue inhaler as set forth in claim 12 wherein said removable cover plate is adapted to be removed from and reinstalled onto said mouthpiece to prevent the infiltration of debris within said passage and said channel.

16. The disposable and compact rescue inhaler as set forth in claim 12 wherein said actuator includes a button and biasing member operatively disposed between said button and said canister-style package to manually actuate delivery of the pressurized fluid medicament into said passage.

17. The disposable and compact rescue inhaler as set forth in claim 12 wherein said actuator includes a locking mechanism having a base and platform substantially defined within said body, said platform adapted to be rotated relative to said base to selectively permit said actuator to engage said canister-style package to prevent unintentional dispensing of the pressurized fluid medicament from said canister-style package.

18. A disposable and compact rescue inhaler for administering fluid medicament in aerosolized form, said inhaler comprising:
a package containing pressurized fluid medicament for treating respiratory constriction;
a housing having an internal cavity defined therein that is adapted to store said package, said housing defined by a plurality of walls wherein at least one of said walls includes an outlet to expel the pressurized fluid medicament from said package in aerosolized form, said housing having a passage disposed between said outlet and said internal cavity to facilitate delivery of the aerosolized medicament toward said outlet, at least one of said walls having at least one inlet defined therein and said housing having at least one channel disposed between said inlet and said passage, said inlet and said channel adapted to direct ambient air into said passage to facilitate aerosolizing the pressurized fluid medicament;
an attachment member operatively attached to said housing and adapted to facilitate removable attachment of said housing to a person;
an actuator having a button recessed within one of said walls and an engaging device that directly engages said package, said button adapted to be manually actuated to direct said engaging device to operatively engage said package to dispense the pressurized fluid medicament into said passage; and
a removable cover plate adapted to fit over said inlet and said outlet to prevent infiltration of debris into said passage and said channel.

19. The disposable and compact rescue inhaler as set forth in claim 18 wherein said package includes a blister-style container having at least one predetermined metered dosage of the pressurized fluid medicament between 130-270 mcg.

20. The disposable and compact rescue inhaler as set forth in claim 18 wherein said housing includes an upper wall, a lower wall, a front wall, a back wall, and first and second sidewalls, said actuator operatively disposed within said upper wall, and a first inlet of said at least one inlet disposed within said first sidewall and a second inlet of said at least one inlet disposed within said second sidewall.
